(19) United States
(12) Patent Application Publication
(10) Pub. No.: US 2024/0203539 A1
(43) Pub. Date: Jun. 20, 2024

(54) MEDICAL DEVICE LINKAGE AND DIAGNOSTIC PERFORMANCE ENHANCEMENT SYSTEM USING MEC AND METHOD USING THE SAME

(71) Applicant: BePlus Healthcare Inc., Gangwon-do (KR)

(72) Inventors: Hoon Jae CHUNG, Seoul (KR); Ki Joon HUGH, Busan (KR); Jae Young KIM, Seoul (KR); Jee Hong KIM, Seoul (KR); Woo Taek LIM, Seoul (KR)

(21) Appl. No.: 18/203,744

(22) Filed: May 31, 2023

(30) Foreign Application Priority Data
Dec. 19, 2022 (KR) 10-2022-0178505

(51) Int. Cl. G16H 10/20; G16H 10/60

(52) U.S. Cl. CPC G16H 10/20; G16H 10/60; G16H 30/20

(57) ABSTRACT

A system includes a database storing a questionnaire scenario, a questionnaire analysis unit analyzing a questionnaire answer from a patient by using an artificial intelligence, a primary diagnosis unit generating a primary diagnosis result by diagnosing the patient based on an analysis result, a medical device linkage unit selecting a necessary test item based on the primary diagnosis result and linked with a medical device, a multi-access edge computing (MEC) linkage unit processing the MEC in linkage with a mobile communication base station, a secondary diagnosis unit generating a secondary diagnosis result by using a test result received from the medical device linkage unit and/or the MEC linkage unit, an electronic medical record (EMR) linkage unit receiving EMR data in linkage with an EMR system, and a diagnostic performance enhancement unit enhancing performance of the questionnaire scenario by using the secondary diagnosis result and the EMR data.

### CROSS REFERENCE TO RELATED APPLICATIONS AND CLAIM OF PRIORITY

This application claims benefit of priority to Korean Patent Application No. 10-2022-0178505, filed on Dec. 19, 2022, in the Korean Intellectual Property Office, the disclosure of which is incorporated herein by reference in its entirety.

### BACKGROUND

### 1. Field

The present disclosure relates to a diagnostic performance enhancement system and a method using the same, and more particularly, to a medical device linkage and diagnostic performance enhancement system using multi-access edge computing (MEC), and a method using the same.

### 2. Description of the Related Art

Questionnaires refer to asking and answering a medical content related to a patient, such as the past history and main symptoms of the patient, and may be the most basic and core data for a diagnosis.

However, the medical data acquired through the questionnaire is typical unstructured data, and it is thus difficult to use the data as digital data.

Therefore, in order to acquire more detailed data from the patient, efforts may be required to generate questionnaire data suitable for a clinic by using an artificial intelligence (AI), and use the same. However, it may take a lot of time and cost to secure dedicated personnel to generate a data set for AI learning in a specialized field such as a medical field.

A conventional questionnaire system simply uses a method of answering yes/no to the questionnaire, and has difficulty in obtaining more detailed data from the patient.

In addition, the conventional questionnaire system based on a computer thinking form may cause a user to feel very awkward and strange by repeating similar questions dozens of times or by providing erroneous questions when there is no conclusion on a question.

Therefore, it is necessary to establish the questionnaire system to prevent repulsion to the user to the user when configured through the artificial intelligence.

In addition, a technology that may quickly process a large amount of data in an emergency is required because the medical data may include such large amounts of data including images.

### SUMMARY

An aspect of the present disclosure is to provide a system which may more quickly perform a diagnosis by processing large amounts of medical image data necessary for a patient in an emergency, and a method using the same.

Another aspect of the present disclosure is to provide a diagnosis system standardizing questionnaires generated through an artificial intelligence to be easily recognized by doctors, and providing a patient with effective treatment counseling, and a method using the same.

Another aspect of the present disclosure is to provide a more accurate diagnosis system by using a diagnosis through a questionnaire and a following test to thus supplement the diagnosis through the questionnaire, and a method using the same.

In an aspect, a medical device linkage and diagnostic performance enhancement system using multi-access edge computing (MEC) may include: a database (DB) storing a questionnaire scenario: a questionnaire analysis unit analyzing a questionnaire answer acquired from a patient by using an artificial intelligence: a primary diagnosis unit generating a primary diagnosis result by diagnosing the patient based on an analysis result of the questionnaire answer that is acquired using the artificial intelligence: a medical device linkage unit selecting a necessary test item based on the primary diagnosis result and linked with a medical device suitable for the selected test item: an MEC linkage unit processing the MEC in linkage with a mobile communication base station: a secondary diagnosis unit generating a secondary diagnosis result by using one or more of a test result received from the medical device linkage unit and a test result received from the MEC linkage unit: an electronic medical record (EMR) linkage unit receiving EMR data in linkage with an EMR system: and a diagnostic performance enhancement unit enhancing performance of the questionnaire scenario by using the secondary diagnosis result and the EMR data.

The medical device linkage unit may be linked with the medical device through short-range mobile communication or internet of things (IOT).

The MEC linkage unit may process medical image data in linkage with the mobile communication base station when the test item is a test item including the medical image data.

The MEC linkage unit may track a patient position.

The diagnostic performance enhancement unit may upgrade the questionnaire scenario to be suitable for the corresponding diagnosis result by referring to the EMR data with the same or similar diagnosis result based on the secondary diagnosis result.

In another aspect, a medical device linkage and diagnostic performance enhancement method using multi-access edge computing (MEC) may include: providing a patient with a questionnaire scenario and analyzing a questionnaire answer acquired from the patient by using an artificial intelligence: generating a primary diagnosis result by diagnosing the patient based on an analysis result of the questionnaire answer that is acquired using the artificial intelligence: selecting a necessary test item based on the primary diagnosis result and performing linkage with a medical device suitable for the selected test item; processing the MEC in linkage with a mobile communication base station in a necessary case: generating a secondary diagnosis result by using one or more of a test result received from the medical device and a test result acquired by processing the MEC: and enhancing performance of the questionnaire scenario by using the secondary diagnosis result and electronic medical record (EMR) data.

In the selecting of the necessary test item based on the primary diagnosis result and the performing of the linkage with the medical device suitable for the selected test item, the linkage may be performed with the medical device through short-range mobile communication or internet of things (IOT).

The necessary case may be a case where the selected test item includes medical image data or a patient position needs to be tracked.

In the enhancing of the performance of the questionnaire scenario by using the secondary diagnosis result and the EMR data, the questionnaire scenario may be upgraded to be suitable for the corresponding diagnosis result by referring to the EMR data with the same or similar diagnosis result based on the secondary diagnosis result in linkage with an EMR system.

As set forth above, the system and the method according to the present disclosure may provide the maximized efficiency of doctor-patient communication through the selected questionnaire before the treatment.

The system and the method according to the present disclosure may provide great help in identifying the patient in remote medical care or in a domestic medical situation where many patients need to be treated in a short time, and reduce misdiagnoses occurring due to insufficient data.

The system and the method according to the present disclosure may assist the doctor to avoid missing an important or serious disease by pre-selecting the medically important questions.

The system and the method according to the present disclosure may be helpful in the early detection and treatment of the serious disease by informing the primary diagnosis, severity, or necessary test and treatment of the patient on behalf of the insufficient medical personnel in the special situation such as the military medical care or the disaster.

The system and the method according to the present disclosure may provide the diagnosis of the patient more quickly and accurately in the emergency by processing the large amounts of medical image data faster.

### BRIEF DESCRIPTION OF THE DRAWINGS

FIG. 1 is a view for explaining a medical device linkage and diagnostic performance enhancement system using multi-access edge computing (MEC) according to an embodiment of the present disclosure.

FIG. 2 is a view showing a configuration of the medical device linkage and diagnostic performance enhancement system using the MEC according to an embodiment of the present disclosure.

FIG. 3 is a view for explaining a medical device linkage and diagnostic performance enhancement method using multi-access edge computing (MEC) according to another embodiment of the present disclosure.

### DETAILED DESCRIPTION OF THE EMBODIMENTS

Various advantages and features of the present disclosure and methods accomplishing the same are apparent from embodiments described below in detail with reference to the accompanying drawings.

However, the present disclosure is not limited to the embodiments described below, and may be implemented in various different forms.

These embodiments in the specification are provided only to make the present disclosure complete and allow those skilled in the art to which the present disclosure pertains to completely appreciate the scope of the present disclosure.

In addition, the present disclosure is defined by the scope of the claims.

Therefore, in some embodiments, well-known components, well-known operations, and well-known techniques are not described in detail in order to avoid ambiguous interpretation of the present disclosure.

In addition, like reference numerals throughout the specification denote like elements, and terms used (or referred to) in the specification are provided for describing the embodiments and are not intended to limit the present disclosure.

In the specification, a term of a singular number may include its plural number unless specifically indicated otherwise in the context, and components and operations referred to as being “included (or provided)” do not exclude the presence or addition of one or more other components and operations.

Unless defined otherwise, all terms (including technical and scientific terms) used in the specification have the same meaning as meanings commonly understood by those skilled in the art to which the present disclosure pertains.

In addition, terms generally used as defined in a dictionary are not to be interpreted as having ideal or excessively formal meanings unless clearly indicated otherwise.

Hereinafter, the embodiments of the present disclosure are described with reference to the accompanying drawings.

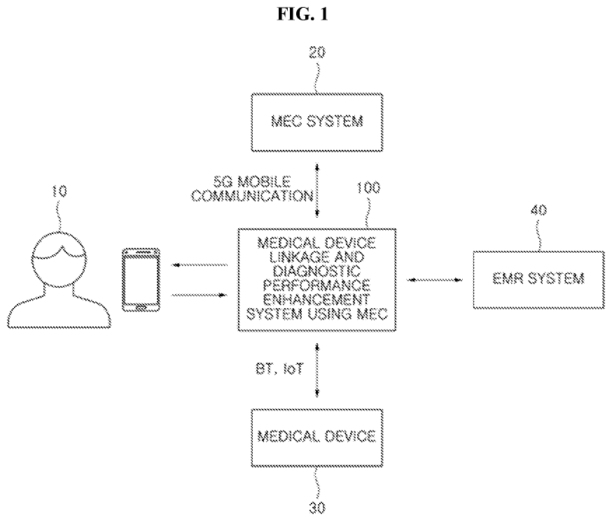

FIG. 1 is a view for explaining a medical device linkage and diagnostic performance enhancement system using multi-access edge computing (MEC) according to an embodiment of the present disclosure.

A medical device linkage and diagnostic performance enhancement system 100 using multi-access edge computing (MEC) according to the present disclosure may receive an answer to a questionnaire item from a patient 10 and analyze the received answer to perform a primary diagnosis of the patient: and select a test item based on the diagnosis and perform a test in linkage with a surrounding medical device 30 to receive a corresponding test result.

In addition, the system 100 may be linked to an electronic medical record (EMR) system 40 storing a diagnosis result and EMR data of the patient to receive the EMR data of the patient, use the diagnosis result and the EMR data to determine whether the diagnosis of the patient 10 is properly performed, and update a questionnaire scenario provided to the patient 10 to perform the diagnosis more quickly and accurately.

Here, the questionnaire scenario indicates a questionnaire set to identify a patient symptom. That is, the questionnaire scenario indicates that a questionnaire suitable for a patient condition is selected and configured as a set in order to identify and analyze the patient symptom and make the diagnosis. In addition, the questionnaire is generated using ‘Korean standard terminology of medicine (KOSTOM)’ and ‘systematized nomenclature of medicine clinical terms (SNOMED-CT)’, and sentences of the questionnaire may thus be expressed with words and sentences suitable for standardization and internationalization.

When provided to the patient, the questionnaire scenario may further use patient data including patient age, education level, area, or the like. Accordingly, the questionnaire scenario may be configured to include a questionnaire item having a sentence expression the patient may easily understand and feel natural.

For example, the expression may be different depending on the patient age, a difficult medical term may be expressed in an easy general term depending on the patient education level, the questionnaire may use the expression in a standard language or a dialect depending on the patient area, or the questionnaire scenario may only include a questionnaire corresponding to the patient data.

Here, the patient data may be acquired from the EMR data of the patient.

The medical device linkage and diagnostic performance enhancement system 100 using MEC according to the present disclosure may quickly process medical image data in linkage with a MEC system 20 through mobile communication when the selected test item is a test including the medical image data.

The medical device linkage and diagnostic performance enhancement system 100 using MEC according to the present disclosure may generate a secondary diagnosis result by using a test result for accuracy of the diagnosis, and perform a more accurate diagnosis based on the test result.

That is, the medical device linkage and diagnostic performance enhancement system 100 using MEC according to the present disclosure may generate the more accurate secondary diagnosis result complementing a primary diagnosis result by using one or more of the received test result in linkage with the medical device 30 and the received test result in linkage with the MEC system 20.

In addition, the medical device linkage and diagnostic performance enhancement system 100 using MEC according to the present disclosure may enhance performance of the questionnaire scenario for the primary diagnosis acquired through a questionnaire analysis to be a more accurate diagnosis by using the more accurate secondary diagnosis result and the EMR data.

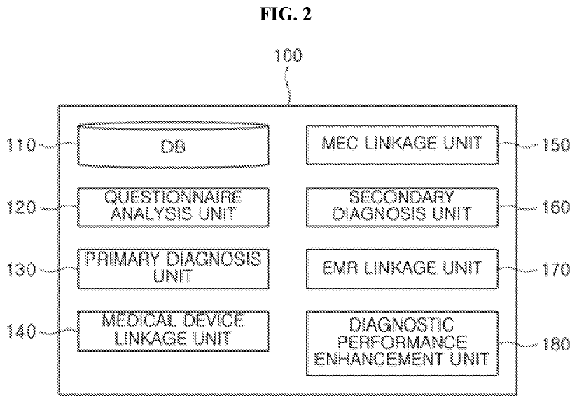

FIG. 2 is a view showing a configuration of the medical device linkage and diagnostic performance enhancement system using the MEC according to an embodiment of the present disclosure.

The medical device linkage and diagnostic performance enhancement system 100 using the MEC according to an embodiment of the present disclosure may include a database (DB) 110, a questionnaire analysis unit 120, a primary diagnosis unit 130, a medical device linkage unit 140, an MEC linkage unit 150, a secondary diagnosis unit 160, an EMR linkage unit 170, and a diagnostic performance enhancement unit 180.

The DB 110 may store the questionnaire item for the diagnosis. The questionnaire scenario is an item set selected to diagnose the patient symptom in consideration of the patient data among the questionnaire items stored in the DB 110.

The questionnaire analysis unit 120 may analyze a questionnaire answer acquired from the patient by using the artificial intelligence.

The questionnaire analysis unit 120 may reconfigure an unclear or ambiguous sentence in which a plurality items exist in one answer to be more concise by structuralizing, standardizing, and encoding contents of the patent questionnaire answers yet to be utilized in actual data analysis because these answers are very important medically but typical unstructured data.

The questionnaire analysis unit 120 may structuralize, standardize, and encode the answers based on its function of analyzing the answer to the questionnaire item.

Structuralizing indicates classifying an attribute necessary for the diagnosis of disease included in the answer to the questionnaire item by meaning.

Standardizing indicates standardizing the attribute classified by meaning into a standardized word or sentence of the same meaning.

Encoding indicates replacing the standardized word or sentence with a code corresponding to the KOSTOM and the SNOMED-CT.

Through such structuralizing, standardizing, and the encoding, the questionnaire analysis unit 120 may analyze the questionnaire answers into encoded data that is more systematic, more standardized, and easily searchable, and store the same, thereby enabling big data analysis in the questionnaire analysis.

The primary diagnosis unit 130 may generate a primary diagnosis result for the patient by diagnosing the patient based on an analysis result of the questionnaire answer that is acquired using the artificial intelligence.

The primary diagnosis unit 130 may evaluate a state of the patient symptom for each attribute through the questionnaire analysis, and diagnose the patient by integrating the states for each attribute.

The medical device linkage unit 140 may select a necessary test item based on the primary diagnosis result and be linked with a medical device suitable for the selected test item.

To this end, the medical device linkage unit 140 may receive a test result for the selected test in linkage with the medical device through short-range communication such as Bluetooth or internet of things (IOT).

For example, the patient may be diagnosed as having a heart disease. In this case, the medical device linkage unit 140 may immediately receive an electrocardiogram result of testing the patient in linkage with a medical device which may measure an electrocardiogram.

Here, the medical device linkage unit 140 may exchange medical data by using health level seven (HL7) and fast healthcare interoperability resource (FHIR) standards to secure its interoperability of a solution with another medical device and establish a data transmission base.

The MEC linkage unit 150 may process multi-access edge computing (MEC) in linkage with a mobile communication base station.

The MEC linkage unit 150 may move traffic and service computing from a centralized cloud to a network edge to quickly process large amounts of data. To this end, the MEC linkage unit 150 may be linked with the mobile communication base station by using mobile communication such as fifth generation (5G).

The selected test item may be the test including the medical image data. In this case, the system 100 may process the medical image in linkage with the MEC system 20 that is installed in the mobile communication base station and performs the edge computing through the mobile communication by the MEC linkage unit 150.

To this end, the MEC linkage unit 150 may analyze and process an X-ray, the electrocardiogram, an ultrasound, a computed tomography (CT) image, or the like in linkage with the MEC system 20 by using the artificial intelligence.

In addition, the MEC linkage unit 150 may track a patient position in linkage with the mobile communication base station.

The MEC linkage unit 150 of the present disclosure may be a hardware or software module and connected to a mobile communication terminal of the patient. Here, the MEC linkage unit 150 may identify a patient current position when the patient 10 accesses the base station through the mobile communication. The secondary diagnosis unit 160 may generate the secondary diagnosis result by using one or more of the test result received from the medical device linkage unit and the test result received from the MEC linkage unit.

The primary diagnosis result is the diagnosis result acquired by analyzing the questionnaire answer, and may thus be an inaccurate diagnosis. Therefore, for a more accurate diagnosis, the secondary diagnosis unit 160 may supplement the primary diagnosis result by using the test result to thus generate the secondary diagnosis result with the added test result.

The EMR linkage unit 170 may receive the EMR data in linkage with the EMR system 40.

The diagnostic performance enhancement unit 180 may enhance the performance of the questionnaire scenario by using the secondary diagnosis result and the EMR data.

The diagnostic performance enhancement unit 180 may upgrade the questionnaire scenario to be suitable for the corresponding diagnosis result by referring to the EMR data of the patient with the same or similar diagnosis result based on the secondary diagnosis result for an accurate diagnosis to be made even only by the questionnaire analysis.

That is, the diagnostic performance enhancement unit 180 may analyze whether a disease predicted by an artificial intelligence algorithm in linkage with the EMR system 40 matches a disease diagnosed by a real doctor to thus correct and upgrade the questionnaire scenario to match an actual diagnosis result even only by the questionnaire analysis.

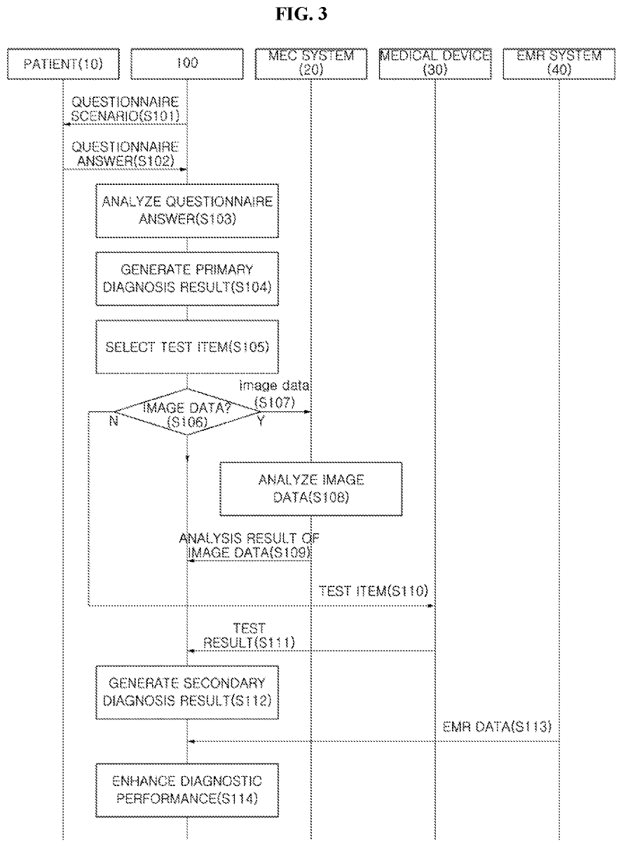

FIG. 3 is a view for explaining a medical device linkage and diagnostic performance enhancement method using multi-access edge computing (MEC) according to another embodiment of the present disclosure.

The method may include providing a patient 10 with a questionnaire scenario by a medical device linkage and diagnostic performance enhancement system 100 using multi-access edge computing (MEC) according to the present disclosure (S101).

The method may include receiving an answer to the questionnaire scenario from the patient 10 (S102). The method may include analyzing the questionnaire answer (S103).

The medical device linkage and diagnostic performance enhancement system 100 using MEC according to the present disclosure may analyze a received answer to a questionnaire item to process the answer sentence by structuralizing, standardizing, and encoding the same.

Structuralizing indicates classifying an attribute necessary for a diagnosis of disease included in the answer to the questionnaire item by meaning: standardizing indicates standardizing the attribute classified by meaning into a standardized word or sentence of the same meaning after structuralizing the answer: and encoding indicates replacing the standardized word or sentence with a code corresponding to ‘Korean standard terminology of medicine (KOSTOM)’ and ‘systematized nomenclature of medicine clinical terms (SNOMED-CT)’ after standardizing the answer.

A database (DB 110) may store a content of the questionnaire, in which the content is divided into a phenomenon (PHENO)/explanation (QUALIFIER) for the attribute of a patient symptom.

The data stored in the DB 110 may be data that is structuralized by performing an operation in which medical personnel input and correct the symptom-related content of the questionnaire.

Therefore, it is possible to acquire data on the symptom indicated by the questionnaire answer when the questionnaire answer is analyzed and the analyzed content is compared and analyzed with data previously worked by the medical personnel, which is stored in the DB 110. Therefore, the diagnosis may be made by the questionnaire analysis.

The method may include generating a primary diagnosis result as a questionnaire analysis result (S104). The method may include selecting a test item based on the primary diagnosis result (S105).

The method may include determining whether the selected test item is a test including medical image data (S106).

The method may include processing the medical image data in linkage with the MEC system 20 when the selected test item includes the medical image data (S107).

The method may include analyzing the medical image data by the linked MEC system 20 (S108).

The method may include receiving an analysis result of the medical image data analyzed by the MEC system 20 (S109).

The method may include processing the selected test item in linkage with a medical device 30 through Bluetooth or internet of things (IOT) when the selected test item includes no medical image data (S110).

The method may include receiving a test result from the medical device 30 (S111).

The method may include generating a secondary diagnosis result by using one or more of the test result received from the medical device and the test result acquired by processing the MEC (S112).

The plurality of test items may exist for the diagnosis, and some of the plurality of test items may include the medical image data, and the others may not include the medical image data.

The method may include receiving electronic medical record (EMR) data in linkage with an EMR system 40 (S113).

The method may include enhancing performance of the questionnaire scenario by using the secondary diagnosis result and the EMR data (S114).

The medical device linkage and diagnostic performance enhancement system 100 using multi-access edge computing (MEC) according to the present disclosure may upgrade the questionnaire scenario to be suitable for the corresponding diagnosis result by referring to the EMR data with the same or similar diagnosis result based on the secondary diagnosis result in linkage with the EMR system 40.

The present disclosure is not limited to the above-described specific embodiments, and may be variously modified by those skilled in the art to which the present disclosure pertains without departing from the scope and spirit of the present disclosure as claimed in the accompanying claims. These modifications should also be understood to fall within the scope and spirit of the present disclosure as claimed in the following claims.

What is claimed is:

1. A medical device linkage and diagnostic performance enhancement system using multi-access edge computing (MEC), the system comprising:
a database (DB) storing a questionnaire scenario;
a questionnaire analysis unit configured for analyzing a questionnaire answer acquired from a patient by using an artificial intelligence;
a primary diagnosis unit configured for generating a primary diagnosis result by diagnosing the patient based on an analysis result of the questionnaire answer that is acquired using the artificial intelligence;
a medical device linkage unit configured for selecting a necessary test item based on the primary diagnosis result and linked with a medical device suitable for the selected test item;
an MEC linkage unit configured for processing the MEC in linkage with a mobile communication base station;
a secondary diagnosis unit configured for generating a secondary diagnosis result by using one or more of a test result received from the medical device linkage unit and a test result received from the MEC linkage unit;
an electronic medical record (EMR) linkage unit configured for receiving EMR data in linkage with an EMR system; and
a diagnostic performance enhancement unit configured for enhancing performance of the questionnaire scenario by using the secondary diagnosis result and the EMR data.

2. The system of claim 1, wherein the medical device linkage unit is linked with the medical device through short-range mobile communication or internet of things (IOT).

3. The system of claim 1, wherein the MEC linkage unit is configured to process medical image data in linkage with the mobile communication base station when the test item is a test item including the medical image data.

4. The system of claim 1, wherein the MEC linkage unit is configured to track a patient position.

5. The system of claim 1, wherein the diagnostic performance enhancement unit is configured to upgrade the questionnaire scenario to be suitable for the corresponding diagnosis result by referring to the EMR data with the same or similar diagnosis result based on the secondary diagnosis result.

6. A medical device linkage and diagnostic performance enhancement method using multi-access edge computing (MEC), the method comprising:
providing a patient with a questionnaire scenario and analyzing a questionnaire answer acquired from the patient by using an artificial intelligence;
generating a primary diagnosis result by diagnosing the patient based on an analysis result of the questionnaire answer that is acquired using the artificial intelligence;
selecting a necessary test item based on the primary diagnosis result and performing linkage with a medical device suitable for the selected test item;
processing the MEC in linkage with a mobile communication base station in a necessary case;
generating a secondary diagnosis result by using one or more of a test result received from the medical device and a test result acquired by processing the MEC; and
enhancing performance of the questionnaire scenario by using the secondary diagnosis result and electronic medical record (EMR) data.

7. The method of claim 6, wherein in the selecting of the necessary test item based on the primary diagnosis result and the performing of the linkage with the medical device suitable for the selected test item,
the linkage is performed with the medical device through short-range mobile communication or internet of things (IOT).

8. The method of claim 6, wherein the necessary case is a case where the selected test item includes medical image data or a patient position needs to be tracked.

9. The method of claim 6, wherein in the enhancing of the performance of the questionnaire scenario by using the secondary diagnosis result and the EMR data, the questionnaire scenario is upgraded to be suitable for the corresponding diagnosis result by referring to the EMR data with the same or similar diagnosis result based on the secondary diagnosis result in linkage with an EMR system.
